SEQUENCE LISTING (1 sequence)

SEQ ID NO: 1 - RNA, 98 nt, Homo sapiens
  source 1..98: mol_type = genomic RNA
cgaggggatacagcagcaattcatgttttgaagtgttctaaatggttcaaaacgtgaggcgctgctataccccctcgtggggaaggtagaaggtgggg

(19) United States
(12) Patent Application Publication
(10) Pub. No.: US 2025/0312285 A1
(43) Pub. Date: Oct. 9, 2025

(54) ALLOGENEIC EXTRACELLULAR VESICLES FOR CANCER TREATMENT

(71) Applicant: Regents of the University of Minnesota, Minneapolis, MN (US)

(72) Inventors: Subbaya Subramanian, Minneapolis, MN (US); Xianda Zhao, Minneapolis, MN (US); Travis J. Gates, Minneapolis, MN (US)

(21) Appl. No.: 19/173,344

(22) Filed: Apr. 8, 2025

Related U.S. Application Data
(60) Provisional application No. 63/575,962, filed on Apr. 8, 2024.

(51) Int. Cl. A61K 9/50; A61K 9/00; A61K 45/06; A61P 35/00; C12N 5/0784

(52) U.S. Cl. CPC A61K 9/5068; A61K 9/0019; A61K 45/06; A61P 35/00; C12N 5/0639

(57) ABSTRACT

The present invention provides compositions of dendritic cells contacted with extracellular vesicles and methods of use thereof. The extracellular vesicles lack or contain a reduce amount of microRNA-424. The dendritic cells may be administered to a subject diagnosed with cancer to treat the cancer or stimulate an anti-tumor response in the subject.

### CROSS-REFERENCE TO RELATED APPLICATIONS

This application claims priority to U.S. Provisional Application No. 63/575,962 filed on Apr. 8, 2024, the contents of which is incorporated by reference in its entirety.

### REFERENCE TO AN ELECTRONIC SEQUENCE LISTING

The contents of the electronic sequence listing (92017100634.xml; Size: 1,886 bytes; and Date of Creation: Apr. 7, 2025) is herein incorporated by reference in its entirety.

### BACKGROUND

Colorectal cancer (CRC) is the third most prevalent cancer diagnosis in the United States, concurrently ranking as the second leading cause of cancer-related mortality. This scenario is further compounded by the escalating CRC incidence observed among patients below the age of 50. The emergence of immune checkpoint inhibitor (ICI) therapies, demonstrated by anti-PD1 and anti-CTLA4, has remarkably broadened therapeutic avenues across diverse malignancies. Nonetheless, the transformative potential of ICIs remains limited to a subset of CRC patients, specifically those harboring the microsatellite instability-high (MSI-H) subtype, constituting less than 15% of the overall CRC population. Remarkably, most CRC patients, >85%, present with a microsatellite stable (MSS) profile, rendering them unresponsive to ICIs. Significantly, the immunogenic differences between the MSI and MSS phenotypes emerged as a major predictive parameter governing the responsiveness to ICIs. The limited occurrence of immune cell infiltration within the subset of MSS-CRC cases further emphasizes the clinical urgency to decipher the intrinsic resistance mechanisms. Therefore, there is a critical need to determine intrinsic resistance mechanisms and increase tumor T cell infiltration to synergize with ICIs.

### SUMMARY

The present invention provides compositions of dendritic cells contacted with extracellular vesicles and methods of use thereof. One aspect of the present disclosure provides a composition comprising a dendritic cell (DC) contacted ex-vivo with an allogeneic extracellular vesicle (EV) to generate an EV loaded DC. In some embodiments, the EV has reduced or lacking expression of microRNA (miRNA)-424. In some embodiments, the EV are exosomes or microvesicles and/or are isolated from a tumor cell that has been modified to inhibit or reduce the expression of miRNA-424. In some embodiments, the tumor cell is a cultured tumor cell or tumor organoid. In some embodiments, the tumor cell or organoid is selected from the group consisting of colorectal cancer cell, breast cancer cell, endometrial cancer cell, prostate cancer cell, lung cancer cell, melanoma cell and pancreatic cancer cell. In some embodiments, the DC is isolated or derived from a subject diagnosed with cancer wherein the DC is derived from a differentiated pluripotent stem cell or myeloid precursor cell isolated from a subject diagnosed with cancer. In some embodiments, the DC are contacted with allogeneic EV isolated from a tumor cell of the same cancer type as the subject with cancer. In some embodiments, the EV comprises additional cargo. In some embodiments, the composition comprises a pharmaceutically acceptable carrier.

Another aspect of the invention provides a method of making an EV loaded DC, the method comprising contacting a DC ex-vivo with allogeneic EV modified to reduce or lack expression of miRNA-424. In some embodiments, the EV is isolated from a tumor cell that has been modified to inhibit or reduce the expression of miR-424, wherein the tumor cell is a cultured tumor cell or tumor organoid. In some embodiments, the tumor cell is selected from the group comprising a colorectal cancer cell, breast cancer cell, endometrial cancer cell, prostate cancer cell, lung cancer cell, melanoma cell and pancreatic cancer cell. In some embodiments the DC is isolated or derived from a subject diagnosed with cancer. In some embodiments, the DC is contacted ex-vivo with allogeneic EV by a method selected from at least one of co-incubation, electroporation, sonication, freeze-thaw, and transfection.

Another aspect of the invention provides a method of treating cancer. The method comprising, isolating a DC from a subject diagnosed with cancer, or deriving a DC from a stem or progenitor cell isolated from a subject diagnosed with cancer, isolating an EV from a tumor cell, wherein the tumor cell is allogeneic to the DC and of the same cancer type as the cancer diagnosed in the subject; wherein the EV has been modified to reduce or lack the expression of miR-424, contacting the DC with the isolated EV ex-vivo to prepare EV loaded DCs, and administering the EV loaded DC to the subject diagnosed with cancer.

Another aspect of the invention provides a method of stimulating an anti-tumor response in a subject having cancer, the method comprising administering any one of the compositions described herein to the subject. In some embodiments, the composition may increase CD28 expression on T cells, increase T cell proliferation, or both. In some embodiments, the anti-tumor response comprises the reduction of tumor growth or inhibition of secondary tumor growth.

### BRIEF DESCRIPTION OF THE DRAWINGS

The patent or application file contains at least one drawing executed in color. Copies of this patent or patent application with color drawing(s) will be provided by the Office upon request and payment of the necessary fee.

The present technology can be better understood by reference to the following drawings. The drawings are merely exemplary to illustrate certain features that may be used singularly or in combination with other features and the present technology should not be limited to the embodiments shown.

FIG. 1: TEV isolation and characterization.
A) Western blot data showing the expression of EV markers, including ALIX (˜100 kDa) and CD81 (˜25 kDa), alongside negative controls β-tubulin (˜60 kDa), and β-actin (˜40 kDa), for MC38 WT, MC38-miR-control, and MC38-424i TEVs.
B) Nanotracker analysis showcasing size distribution profiles of TEVs derived from MC38-424i (yellow), MC38-miR-control (red), and MC38-WT (black) TEVs.
C) Transmission electron microscopy images presenting the morphological characteristics of MC38-WT, MC38-miR-control, and MC38-424i TEVs.

FIG. 2: Treatment with allogeneic MC38 TEVs on CT26 tumor slows tumor progression.
A) Schematic representation illustrating the administration of TEVs, tumor inoculation, and images capturing tumor progression within distinct groups (Saline n=8; MC38-WT TEV n=7; MC38-miR-control TEV n=7; MC38-424i TEV n=9).
B) Flow cytometry validation confirming the depletion of α-CD4 and α-CD8 T cells, accompanied by tumor images post TEV inoculation in BALB/c mice bearing CT26 tumors.
C) Comparison of endpoint tumor volumes among different groups (saline, MC38-WT TEV, MC38-miR-control TEV, MC38-424i TEV, MC38-424i TEV+α-CD4, MC38-424i TEV+α-CD8), respectively. (*** p<0.005) (Error bars+/−SEM).

FIG. 3: Treatment with allogeneic MC38 TEVs modulates T cell infiltrates:
A) Immunofluorescence depicting CD8⁺ T cell distribution (bright) and DAPI nuclear staining within CT26 tumors, comparing saline-treated (bright) and MC38-424i TEV-treated groups. (* p<0.05) (Error bars+/−SEM).
B) Peripheral blood cytokine levels from mice treated with saline or MC38-424i TEV.
C) Quantification of cytokine levels in peripheral blood, contrasting saline-treated (closed circle) and MC38-424i TEV-treated (open circle) groups. Error bars+SEM.

FIG. 4: Treatment with MC38 TEVs on B16 melanoma tumors.
A) Schematic representation of the administration of MC38 TEVs in C57BL/6J mice bearing B16-F10 tumors.
B) Comparison of tumor volumes between the saline-treated group (black) and the MC38-424i TEV-treated group (grey). (ns p>0.05) (Error bars+/−SEM).
C) Immunofluorescence visualization of CD8⁺ T cells (red) and DAPI nuclear staining within B16-F10 tumors, comparing the saline-treated group and the MC38-424i TEV-treated group. (ns p>0.05) (Error bars+/−SEM).

FIG. 5: Dendritic cell isolation and TEV capture in vitro.
A) Comparison of dendritic cell morphology on day 6 with (left) and without (right) differentiation induced by IL4, TNFα, GM-CSF, and LPS.
B) Mean fluorescence intensity of MHCII-APC-Cy7 in naïve splenocytes and day 6 in vitro differentiated dendritic cells (DCs).
C) Fluorescence microscopy images demonstrating the uptake of TEVs (red) by DCs, with and without labeling of TEVs using DiO (green). Nuclei stained with DAPI are shown in blue. A cross-sectional view (XZ-plane) of Z stack images containing DiO-labeled TEVs is also presented.

FIG. 6: Autologous transfer of DCs exposed to TEVs slows tumor growth.
A) Illustration depicting the process of dendritic cell isolation, TEV pulsing, and autologous transfer of DCs to BALB/c animals before tumor challenge with CT26 colon cancer cells.
B) Images showing the tumor status at the experimental endpoint for the MC38-424i, MC38 WT, MC38-miR-control, No TEV, and saline groups (n=5/group).
C) Graph depicting the tumor volumes at the experimental endpoint for the MC38-424i, MC38-WT, MC38-miR-control, no TEV, and saline groups (n=5/group). (* p<0.05) (ns p>0.05) (Error bars+/−SEM).
D) Immunofluorescence images and quantification of CD8⁺ T cells (blue) and DAPI-stained nuclei in CT26 tumors among the saline, MC38-WT, and MC38-424i TEV treatment groups. (* p<0.05) (ns p>0.05) (Error bars+/−SEM).

FIG. 7: Fluorescence microscopy images demonstrating the uptake of labeled DiO TEVs (green) by DCs (red). Nuclei stained with DAPI (blue).

### DETAILED DESCRIPTION

The present invention provides compositions of dendritic cells contacted with extracellular vesicles and methods of use thereof. The inventors demonstrated the capacity of dendritic cells (DCs) to internalize tumor-derived extracellular vesicles (EV) and a possible mechanism to elicit an anti-tumor immune response using these contacted or loaded DCs. In particular, the inventors demonstrate autologously administered DCs, which had been exposed to allogeneic EV which lack immune-suppressive miR-424, elicit a robust CD8⁺ T cell response and limit tumor growth in a colorectal cancer model.

### Compositions:

In a first aspect, the present invention provides a composition comprising a dendritic cell (DC) contacted ex-vivo with an allogeneic extracellular vesicle (EV) to generate an EV loaded DC, wherein the EV has reduced or lacking expression of microRNA (miRNA)-424.

Dendritic cells (DCs) are a heterogeneous population of myeloid immune cells characterized by phagocytic and antigen presentation capacity. Although DCs are categorized as innate immune cells, they are responsible for initiating adaptive immune responses, in particular, for the antigen-specific activation of naive T cells. Activated DCs show increased antigen uptake, migratory capacity, and ability to prime naive T cells in lymph nodes. Circulating DC can be conventional DC, myeloid DCs and plasmacytoid DC. DCs are also present in tissues, in particular in tissues that are in contact with the external environment, such as the skin, lining of the nose, lung, stomach and intestines.

DC can be isolated by any means known in the art. Typical methods of isolation comprise the isolation of DC from peripheral blood, or the isolation of stem or progenitor cells from peripheral blood, which are differentiated ex-vivo into DC. A typical process of isolating DC from peripheral blood comprises, the preparation of a buffy coat from a peripheral blood sample, then the buffy coat, or whole blood can be subjected to density gradient centrifugation to isolate mononuclear cells. The mononuclear cells can then be subjected to a selection process by which DC are specifically selected for, or alternatively, cells other than DC are selectively removed. Various commercially available kits are available for this process. Methods for separation of DC from other blood cells, include, but are not limited to, immunomagnetic cell separation, fluorescence-activated cell sorting, leukapheresis, density gradient centrifugation, immunodensity cell isolation, microfluidic cell sorting, buoyancy-activated cell sorting and aptamer-based cell isolation. Markers of DC include, but are not limited to HLA-DR, CD1a, CD1c, CD11c, CD11b, CD141, CD123, CD209, CD303, CD304, BATF3, IRF8, IRF2, IRF4, RelB, RBP-J. In methods for the depletion of cells other than DC, cells which express CD3, CD14, CD16m CD19, CD83, CD56 and glycophorin A are depleted from a sample to enrich for DC. Kits for the isolation or enrichment of DC can be used on various biological samples, including but not limited to whole blood, plasma, buffy coat, and umbilical cord blood.

DC can also be derived by the differentiation of stem, progenitor and induced pluripotent cells or myeloid precursor cell into DC ex-vivo. A derived dendritic cell may be differentiated or matured from a different cell type, such as a stem or progenitor cell, through the use of transcription factors, growth factors or other factors such that the cell acquires characteristics of a dendritic cell. Differentiation protocols are known in the art and comprise culturing cells in various cytokines and growth factors to differentiate the cells into DC. These cytokines and growth factors include, but are not limited to BMP-4, VEGF, SCF, GM-CSF, IL-4, Flt3L, TNF-α, IL-7, IL-3. Some protocols use the induction of transcription factors including, but not limited to IRF8, PU.1, E2-2. In some embodiments of the present invention, DC are isolated or derived from a subject.

The DC can be isolated or derived by the methods described herein, and those known in the art. In some embodiments, the subject has cancer. Cancer is a term for diseases in which abnormal cells divide without control and can invade nearby tissues. Cancer cells can also spread to other parts of the body through the blood and lymph systems. The term “subject” may be used interchangeably with the terms “individual” and “patient” and includes human and non-human mammalian subjects. A “subject in need thereof” as utilized herein may refer to a subject in need of treatment for a disease or disorder associated with a suspected tumor or cancer. A tumor or cancer may include, but is not limited to bladder cancer, bone cancer, brain cancer, breast cancer, cervical cancer, colon cancer, esophageal cancer, gastric cancer, head & neck cancers, Hodgkin's lymphoma, leukemia, liver cancer, lung cancer, melanoma, mesothelioma, multiple myeloma, myelodysplastic syndrome, non-Hodgkin's lymphoma, ovarian cancer, pancreatic cancer, prostate cancer, rectal cancer, renal cancer, sarcoma, skin cancer, testicular cancer, thyroid cancer, uterine cancer and any other cancer or solid tumor. In some embodiments, a subject is diagnosed with a cancer, wherein the cancer comprises colorectal cancer, breast cancer, endometrial cancer, prostate cancer, lung cancer, melanoma and pancreatic cancer; and a DC is isolated or derived from the subject.

The composition described herein comprises a DC contacted ex-vivo with an allogeneic extracellular vesicle (EV). EVs are cell-derived membrane-surrounded vesicles that can carry bioactive molecules which can be delivered to recipient cells. Classical EVs are exosomes, microvesicles, and apoptotic bodies. EV are heterogeneous in size ranging from 20 nm-10 um and their content may comprise lipids, RNA, DNA protein, or other such cargo. Microvesicles are a type of extracellular vesicle that are released from the cell membrane and can vary in size between 50 nm and 1000 nm. Extracellular vesicles isolated from a tumor cell can be called tumor-derived extracellular vesicles (TEV). Methods of isolating EV from a cell are known in the art and include, but are not limited to differential centrifugation, immunomagnetic separation, microfluidic based isolation, charge-based isolation, size-based isolation, affinity chromatography, and antibody-based isolation.

An allogeneic EV is from the same species as the DC used herein, but a different individual. Typically, allogeneic cells or tissues are genetically dissimilar and immunologically incompatible. In some embodiments, the allogeneic EV are derived from a tumor cell or tumor organoid, including, but not limited to colorectal cancer cell, breast cancer cell, endometrial cancer cell, prostate cancer cell, lung cancer cell, melanoma cell and pancreatic cancer cell. An allogeneic EV may be derived from a tumor cell of the same tumor type as the subject, but not from a tumor cell of the subject. The cells used to produce the EVs may be tissue culture cell lines derived from a cancer. Thus, the EV is allogeneic to the subject from whom the DC are isolated.

In some embodiments the EV has reduced or lacking expression of microRNA (miRNA)-424. miRNAs are short (20-24 nt) non-coding or coding RNAs that are involved in post-transcriptional regulation of gene expression in multicellular organisms by affecting both the stability and translation of mRNAs. miRNA-424 is a member of the family of miR15/107. Members of this family have AGCAGC sequences in the Seed area and are involved in the cell division, apoptosis, stress responses, and cancer. Human miRNA-424 has the following sequence:

(SEQ ID NO: 1)
CGAGGGGAUACAGCAGCAAUUCAUGUUUUGAAGUGUUCUAAAUGGUUCAA
AACGUGAGGCGCUGCUAUACCCCCUCGUGGGGAAGGUAGAAGGUGGGG.

The role of miRNA-424 in cancer is unclear as it has been shown to be upregulated in some cancer types and downregulated in other cancer types.

In some embodiments, the EV is derived from a tumor cell with reduced or lacking miRNA-424. A tumor cell may be modified in any way which decreases or eliminates the expression or function of miRNA-424. Methods of reducing or eliminating miRNA-424 are known in the art, and may comprise traditional recombinant biology methods of genetic modification and may include, but are not limited to genetic modification such as genetic mutation, homologous recombination, non-homologous end-joining, CRISPR-Cas9 mediated genetic editing, and Talen based methods or pharmacologic methods. In some embodiments, steric blocking may be used, that is using an oligonucleotide that is complementary to the mature miRNA target. miRNA inhibitors form a duplex with the miRNA guide strand that prevents the miRNA from binding to its intended target. In some embodiments an oligonucleotide complementary to miRNA-424 may be used such that it inhibits or decreases the expression of miRNA-424 in the tumor cell and thus the tumor cell produces EV that are lacking or have reduced miRNA-424. In some embodiments, a tumor-derived EV may be modified to inhibit or reduce the expression of miRNA-424. For example, siRNA may be electroporated into a tumor-derived EV, wherein the siRNA is complementary to miRNA-424 such that the expression of miRNA-424 is decreased or reduced. Tumor derived EV may be transfected or electroporated with oligonucleotides or other small molecules to inhibit or reduce the expression of miRNA-424.

In some embodiments, the EV may also contain additional cargo or a cargo molecule. A cargo molecule may comprise any molecule which is to be transported or delivered by the EV. By way of example and not limitation EV cargo may comprise RNA, DNA, active pharmaceutical ingredients, anticancer drugs, small molecules, adjuvants, proteins, therapeutic cargo, gene editing cargo such as Cas9, delivery of vaccines including mRNA and/or adjuvants. Cargo may be organ, tissue or cell type specific.

In some embodiments, a DC is contacted ex-vivo with an allogeneic EV. “Contacting” as used herein, refers to contacting a DC directly or indirectly. Contacting a cell includes adding an agent to a cell in-vitro or ex-vivo. The contacting may comprise culturing the DC in culture medium comprising the EV. The DC should be contacted for a sufficient time and under sufficient conditions to allow the DC to uptake the EV. The DC may essentially be transfected by the EV by contacting the DC for a sufficient time and under sufficient conditions to allow the contents of the EV to be delivered to the DC. The DC may be contacted specifically or non-specifically by the EV. For example, the EV may contact the DC through specific surface receptors or by diffusion. In some embodiments, the DC is contacted by a method selected from co-incubation, electroporation, sonication, freeze-thaw, and transfection. When an EV contacts a DC, the contents of the EV is delivered to the DC. This may occur through endocytosis, macropinocytosis, phagocytosis, lipid raft-mediated uptake or direct membrane fusion. A DC that has been contacted by an EV, as described herein, maintains its functionality as an antigen presenting cell and its expression of CD80, allowing for interactions with T lymphocytes.

The compositions described herein may be administered to a subject by any means known in the art. As used herein, the terms “administering” and “administration” refer to any method of providing a composition described herein, or a pharmaceutical preparation thereof to a subject comprising the loaded DC described herein. Such methods are well known to those skilled in the art and include, but are not limited to, transdermal administration, administration by inhalation, nasal administration, and parenteral administration, including injectable such as intramuscular administration, intradermal administration, intravenous and subcutaneous administration.

In another aspect, the present disclosure provides pharmaceutical compositions comprising one or more of the compositions as described herein and an appropriate carrier, excipient or diluent. The exact nature of the carrier, excipient or diluent will depend upon the desired use for the composition and may range from being suitable or acceptable for veterinary uses to being suitable or acceptable for human use. The composition may optionally include one or more additional compounds.

In some embodiments, the EV have been modified to comprise additional cargo. In some embodiments, the additional cargo comprises proteins, small molecules and/or nucleic acids. Any of these may be or include an anti-cancer drug, an immunostimulatory mediator or other immunotherapeutics. By way of example and not limitation anti-cancer drugs may include chemotherapy drugs, targeted therapies, hormonal agents, antimetabolites, and or immunotherapeutics. One exemplary cargo that may be administered as part of the loaded DC composition is an immune checkpoint inhibitor (ICI). ICI including but not limited to antibody-based inhibitors of PD-1, PDL-1, or CTLA-4 are known in the art and may be included in the compositions provided herein.

As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

In another embodiment, the present formulation may also comprise other suitable agents such as a stabilizing delivery vehicle, carrier, support or complex-forming species. The coordinate administration methods and combinatorial formulations of the instant invention may optionally incorporate effective carriers, processing agents, or delivery vehicles, to provide improved formulations for delivery of the loaded DC described herein.

The formulation may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final formulation.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservative.

### Methods:

In a second aspect, the present disclosure provides a method of making an EV loaded DC, the method comprising contacting a DC ex-vivo with an allogeneic EV modified to reduce or lack the expression of miRNA-424. In some embodiments, the method comprises a) isolating a DC from a subject diagnosed with cancer, or deriving a DC from a stem or progenitor cell isolated from a subject diagnosed with cancer; b) isolating an EV from a tumor cell, wherein the tumor cell is allogeneic to the DC and of the same cancer type as the cancer diagnosed in the subject, and wherein the EV has been modified to reduce or lack the expression of miRNA-424; and c) contacting the DC of step a) with the isolated EV of step b) ex-vivo to prepare EV loaded DCs. The EV loaded DCs made by the method may be used to treat a cancer by administering the EV loaded DC of step c) to the subject diagnosed with cancer. The DC and the EV should be derived from a subject with the same type of cancer as the cancer cells from which the EV are isolated. Thus, for treating a subject with colorectal cancer the EVs should be isolated from a colorectal cancer cell line and for treating a subject with breast cancer, the EVs should be isolated from a breast cancer cell line. In the contacting step of the method the EVs may be simply added to the culture medium containing the DCs and allowed to incubate for a period of time to allow for co-incubation or the DCs and EVs may be electroporated, sonicated or subject to freeze-thaw or transfection conditions to increase interactions of the EVs with the DCs.

In some embodiments, the EV is further modified to comprise additional cargo as discussed above. In some embodiments, a range of DC are administered to the subject. The range of DC may comprise an effective amount of DC and may comprise 0.5 x10⁶ to about 1x 10⁶ DC per injection. Suitable ranges for a single administration may be from 10⁵ to 108 or 5×10⁵ to 10⁷ or any amount between these ranges. The loaded DC may be administered by any means known in the art, including, but not limited to intravenously, intratumorally, subcutaneously, or intraperitoneally.

Another aspect of the present disclosure provides a method of treating cancer. In some embodiments, the method comprises a) isolating a DC from a subject diagnosed with cancer, or deriving a DC from a stem or progenitor cell isolated from a subject diagnosed with cancer; b) isolating an EV from a tumor cell, wherein the tumor cell is allogeneic to the DC and of the same cancer type as the cancer diagnosed in the subject; wherein the EV has been modified to reduce or lack the expression of miR-424; c) contacting the DC of step (a) with the isolated EV of step (b) ex-vivo to prepare EV loaded DCs; and d) administering the EV loaded DC of step (c) to the subject diagnosed with cancer. The subject may be administered the loaded DCs provided in as a composition to treat the cancer. The subject may be administered the composition provided herein with other anti-cancer agents, such as an ICI and these agents may be administered before at the same time as or after the loaded DC compositions. If administered at the same time, the anti-cancer agent may be added to the loaded DCs as an additional cargo.

In some embodiments, the DC is contacted ex-vivo with the allogeneic EV via co-incubation, electroporation, sonication, freeze-thaw, or transfection. In some embodiments, a range of DC are administered to the subject. The range of DC may comprise an effective amount of DC and may comprise 0.5×10⁶ to about 1×10⁶ DC per injection. In some embodiments, at least 10⁶ loaded DC are administered to the subject. In some embodiments, the loaded DC are administered intravenously, intratumorally, subcutaneously, or intraperitoneally.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. For example, treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer. In some embodiments, the optimum effective amount can be readily determined by one skilled in the art using routine experimentation.

Another aspect of the present disclosure provides a method of stimulating an anti-tumor response in a subject having cancer, the method comprising, administering a composition described herein. In some embodiments, the method increases CD28 expression on T cells and/or increases T cell proliferation. CD28 is a protein expressed on T cells that provides co-stimulatory signals required for T cell activation and survival. In some embodiments the anti-tumor response may reduce, repress, delay or prevent cancer growth, reduce tumor volume and or prevent, repress, delay or reduce metastasis of the tumor. An anti-tumor response may also reduce the number of tumor cells within the subject.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

### Example 1

In the following example, the inventors describe the capacity of dendritic cells (DCs) to internalize TEVs, and a possible mechanism to elicit an anti-tumor immune response. Moreover, the inventors’ investigation of autologously administered DCs, which had been exposed to MC38 modified TEVs, underscores their potential to dampen tumor growth while elevating CD8⁺ T cell levels vis-à-vis MC38 wild-type TEVs exposed to DCs. The inventors' findings collectively underscore the potential of allogeneic-modified TEVs without immune-suppressive miR-424 to elicit robust CD8⁺ T cell responses and limit tumor growth in CRC.

### Results:

### Allogeneic Modified TEVs Increase T Cell Infiltrates and Decrease Tumor Growth

Previously, we demonstrated that TEVs derived from CRC cells contain immunosuppressive miR-424, which impacts T cell co-stimulation and hinders the effectiveness of ICIs.¹⁹ We aimed to investigate the potential impact of allogeneic modified TEVs (TEVs characterized by the absence of functional miR-424) derived from MC38 colon cancer cell lines of a C57BL/6 background on BALB/c mice harboring CT26 cell-based tumors. To test this, we used MC38 wild-type (MC38-WT) cell lines and modified MC38 cell lines that stably express miR-424 inhibitor (MC38-424i) and MC38-424i scramble control (MC38 miR-control). First, we confirmed the quality of TEVs isolated from each cell line by western blotting and determined the EV markers CD81, and ALIX. We also included b-actin and b-tubulin to ensure no cellular contaminants were present in TEV isolation (FIG. 1A). The presence of ALIX and CD81 was evident in MC38-WT, MC38-424i, and MC38-miR-control groups, while b-actin and b-tubulin were not observed. No positive signal was detected despite the potential for b-actin presence in TEV cytoskeletal components.

Subsequently, we conducted Nanotracker analysis to determine the size distribution across the MC38-WT, MC38-424i, and MC38-miR-control TEVs (FIG. 1B). Notably, the average size distributions of TEVs were determined to be 136.9, 144.8, and 97.1 nm for MC38-WT, MC38-424i, and MC38-miR-control TEV groups, respectively. Transmission electron microscopy imaging gained further insight into TEV morphology, which provided additional validation of the TEV purity and structure (FIG. 1C).

To investigate the potential impact of allogeneically modified TEVs on tumor growth, we prophylactically administered BALB/c mice with two injections of 10 mg each of MC38-WT, MC38-424i, MC38-miR-control TEVs, or saline, as illustrated in FIG. 2A. A period of 10 days was allowed to develop an adaptive immune response, after which the mice were challenged with CT26 cells at a dose of 2×10⁵ cells per injection. After inoculation, a 21-day interval was permitted to allow for tumor progression. Mice treated with MC38-424i TEVs displayed strikingly smaller tumors (71.49±30.32 mm³) in comparison with the saline group (380.9±95.48 mm³), MC38-WT group (284.7±108.1 mm³), and MC38-miR-control group (352.3±128.5 mm³). Notably, within the MC38-424i group, three mice exhibited complete tumor regression following the CT26 tumor challenge, as shown in FIG. 2A.

Furthermore, we sought to determine whether the observed tumor phenotype was contingent on the presence of CD4 or CD8 T cells. To ascertain this, we administered two intraperitoneal injections of depletion antibodies, targeting aCD4 and aCD8 (at a dose of 400 mg per injection), before administering allogeneic TEVs. The efficacy of CD4⁺ and CD8⁺ T cell depletion was evaluated using flow cytometry, comparing the depleted mice to naive spleen and lymph nodes 3 and 10 days after CD4⁺ and CD8⁺ depletion, as represented in FIG. 2B. After implementing the experimental regimen outlined in FIG. 2A, we confirmed that the depletion of CD4⁺ and CD8⁺ T cells in BALB/c mice compromised the influence of allogeneically modified TEVs on CT26 tumors. Specifically, MC38-424i and anti-CD4-treated mice exhibited augmented tumor volumes (791.5±85.99 mm³), and MC38-424i and anti-CD8-treated mice displayed a similar trend (619.5±151.5 mm³), as shown in FIG. 2C. Therefore, we concluded that MC38-424i TEVs substantially inhibited tumor growth compared with MC38-WT, MC38-miR-control TEVs, and saline control groups. Furthermore, this observed phenotype appeared contingent on CD4⁺ and CD8⁺ T cells in vivo.

Treatment with Allogeneic Modified TEVs Increases Tumor-infiltrating T cells

Following the convincing observation of a substantial impact on tumor growth within the context of allogeneic MC38-424i TEVs in comparison with MC38-WT TEVs, MC38-miR-control TEVs, and the saline control groups, we proceeded with immunofluorescence analysis of tumor tissues. The objective was to determine the potential differences in T cell infiltration between CT26 tumors treated with allogeneic MC38-424i TEVs and those treated with saline. Our analysis revealed a significant difference in CD8⁺ cell counts per field, registering at 33.11±7.21 and 17.17 ±3.42 for allogeneic MC38-424i TEVs and saline treatments, respectively (FIG. 3A).

Considering the broad-ranging influence of TEVs on cellular responses and the distinct genetic background of MC38-424i TEVs, we considered it key to investigate the safety profile of MC38-424i TEVs compared with saline. We assessed peripheral blood cytokines using a protein array, including tumor necrosis factor alpha (TNF-α), IL-6, IL-2, IL-10, and IFN-g. Notably, our analysis indicated the absence of a significant increase in the detected cytokines between the allogeneic MC38-424i TEV and saline groups (FIG. 3B).

### Allogeneic Modified TEVs Do Not Significantly Influence B16-F10 Tumor Growth

After observing a noticeable influence of MC38-424i TEVs on both CT26 colon cancer growth and T cell infiltration, we examined the specificity of this effect within the context of CRC tumor models. We adopted an analogous prophylactic model to investigate this, administering MC38-424i TEVs to C57BL/6 mice challenged with B16-F10 melanoma cells FIG. 4A. Mice were administered two pro-phylactic doses (10 mg per injection) of allogeneic MC38-424i TEVs or saline on day 1 and day 4. On day 10, C57BL/6 mice were subjected to a subcutaneous injection containing 2 10⁵ B16-F10 melanoma cells. Remarkably, we did not observe a significant difference in endpoint tumor volumes, measuring 1,386±536.1 and 1,657±187.5 mm³ for the allogeneic modified TEV and saline groups, respectively (FIG. 4B). However, substantial variability in tumor volume was evident within the allogeneic MC38-424i TEV group. In addition, analysis of CD8⁺ cell counts per field indicated no significant differences, revealing 12.8±3.1 and 15.6±3.3 for the allogeneic MC38-424i TEV and the saline groups, respectively (FIG. 4C).

### Allogeneic TEVs Pulsed Dendritic Cells are Instrumental in Stimulating an Anti-Tumor Immune Response

Upon observing the tumor-specific activation of the immune response induced by allogeneic modified TEVs, we investigated the mechanistic underpinnings of TEV processing in an in vitro setting. We hypothesized that DCs capture and present TEVs, thereby enabling the exhibition of tumor antigens within these TEVs to T cells. This presentation could consequently elicit an anti-tumor immune response. Such a phenomenon could also explain the observed indirect detriment to the phenotype upon depletion of CD4⁺ and CD8⁺ T cells. To assess the plausibility of TEV capture by DCs, we isolated monocytes and fostered in vitro differentiation into DCs by introducing Il-4, GM-CSF, TNF-a, and LPS. Subsequently, we performed imaging of the DC populations over 6 days, both in the presence and absence of differentiation factors (FIG. 5A).

Furthermore, we conducted flow cytometric analysis, confirming the expression of MHC class II between enriched DCs (mean signal intensity of 1,993) and undifferentiated monocytes sourced from naive spleen and lymph nodes (mean signal intensity of 374) (FIG. 5B). Subsequently, we carried out an in vitro experiment designed to assess the up-take of TEVs. TEVs were stained with the lipophilic dye DiO and introduced to DCs cultured on fibronectin-coated glass slides (5 mg/mL). Following a 24-h incubation period, we proceeded to visualize the outcome. Remarkably, we observed the intracellular localization of the DiO signal (green) within the X-Z plane of the DCs that had been stained with Cytopainter Red (FIG. 5C).

Having established that DCs can capture MC38 TEVs, we examined the potential of allogeneic MC38-424i TEVs pulsed onto DCs derived from a BALB/c background in cultured conditions to confer protection against CT26 tumor challenge. This approach could enable the targeted delivery of TEVs without necessitating their direct administration into the bloodstream. To evaluate this strategy, we enriched DCs from the spleen of BALB/c mice and subjected them to MC38-WT, MC38-miR-control, and MC38-424i TEVs on the sixth day of DC differentiation. The following day, we autologously transferred 1×10⁶ DCs exposed to TEVs via intravenous tail vein injection. Our experimental design encompassed five groups of BALB/c mice (n=5/group): MC38 WT TEV, MC38-424i TEV, MC38-miR-control TEV, no TEV, and saline. After allowing a 14-day interval following DC administration, we introduced a CT26 tumor challenge, allowing tumor progression over 21 days (FIG. 6A).

Substantial variations in tumor volumes were evident when comparing all TEV groups with both DCs without TEVs (1,105±37.4 mm³) and saline (1,186.4±25.2 mm³) groups, showing a significant difference.

Intriguingly, no notable differences in tumor volumes were observed among the MC38-WT TEV (834.4±65.4 mm³), MC38-miR-control TEV (910.6±84.4 mm³), and MC38-424i TEV (655.8±355.4 mm³) groups (FIGS. 6B and 6C). Notably, MC38-424i TEVs (655.8±355.4 mm³) revealed a significant difference in tumor volumes compared with the no TEVs (1,105 ±37.4 mm³) and saline groups (1,186.4±25.2 mm³).

Despite the absence of noticeable differences in tumor volumes across the TEV groups, we investigated CD8⁺ T cell infiltrates. Remarkably, we observed significant variations in CD8⁺ cell counts per field. We detected 29.4±8.6, 18.0±5.2, and 16.4±4.0 for the MC38-424i TEV, MC38 WT TEV, and saline groups, respectively (FIG. 6D).

### DISCUSSION

This study focused on understanding the consequences of administering MC38 allogeneic TEVs, which lack functional miR-424, on tumor growth dynamics and T cell infiltrates in mice harboring CT26 or B16-F10 tumors. We hypothesized that the administration of MC38-424i TEVs would control the growth of both CT26 and B16-F10 tumors.

Substantiating this hypothesis, we observed a substantial reduction in tumor volumes upon the prophylactic administration of MC38-424i TEVs to mice subjected to CT26 tumor challenges (FIGS. 2A and 2C). However, in the context of C57BL/6 animals challenged with B16-F10 tumors, we did not observe any differences in endpoint tumor volumes following the administration of MC38-424i TEVs (FIG. 4B). Despite this, two animals displayed diminished tumor sizes at the experimental endpoint; nevertheless, the group variability did not yield statistically significant outcomes.

It has been reported that conserved tumor exon junctions between MC38 cells and B16-F10 cells can be presented on MHC class I.28 Furthermore, prophylactic exposure to these conserved tumor exon junctions has been reported to confer protective effects against B16-F10 growth, attributed to elicited anti-tumor immune responses.²⁸ Given these findings, we speculate that conserved tumor neoantigens present within MC38 TEVs and B16-F10 melanoma cells might undergo immunoediting, ultimately facilitating immune evasion.^{29,30}

On the contrary, the tumor neoantigens shared between MC38 TEVs and B16-F10 tumors might evoke a comparatively weak immune response.³¹ Notably, existing literature highlights that MC38 cells and CT26 cell lines bear a comparable load of somatic mutations per megabase, resulting in a similar tumor mutational burden.³² These two observations collectively explain the divergent outcomes observed in our study. Specifically, the robust immune response (FIG. 3A) and the conferred protective effects on tumor growth (FIG. 2C) witnessed with MC38-424i TEVs in CT26 tumors can be attributed to these factors, whereas a parallel immune response was not attained in B16-F10 tumors. Similarly, we documented a substantial augmentation in CD8+T cells within CT26 tumors following miR-424i TEV treatment (FIG. 3A), whereas no corresponding increment was discerned in CD8⁺ T cells within B16-F10 tumors receiving the same treatment (FIG. 4C). These observations underscore that the origin of tissue or similarities in tumor mutational burden could play a pivotal role in determining the efficacy of allogeneic TEVs. This is substantiated by the observation that the impact of MC38-424i TEVs on B16-F10 immune infiltrates and tumor growth was relatively subdued.

Furthermore, it is noteworthy that no significant increase in peripheral blood cytokines was observed upon comparing allogeneic MC38-424i TEVs with saline. These data support that the administration of TEVs was well tolerated within an allogeneic setting (FIG. 3B). This observation is significant, as any induction of a cytokine release syndrome profile could undermine the clinical translatability of allogeneic TEVs.33,34

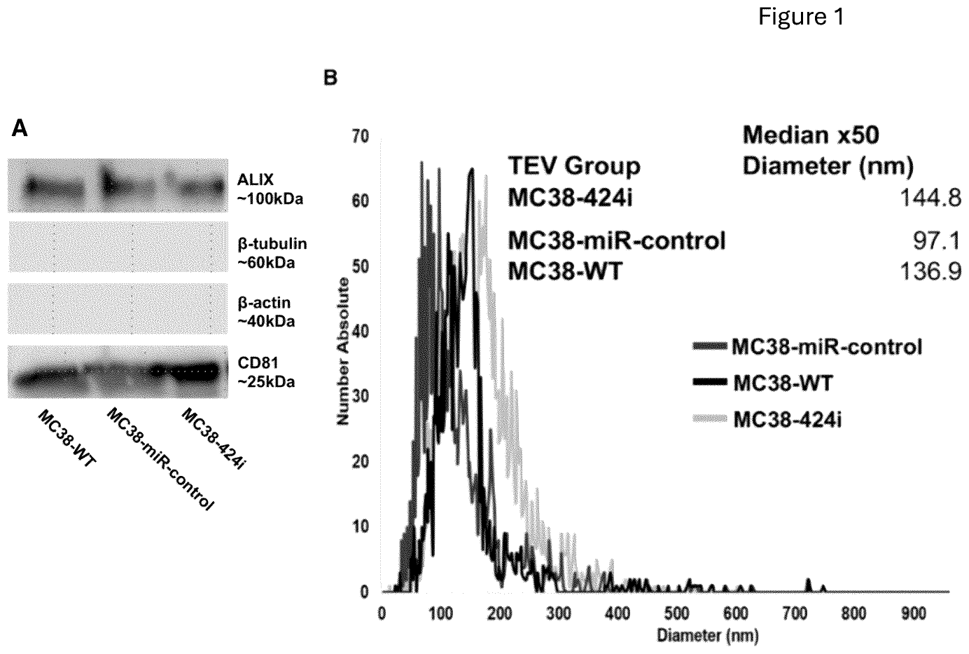

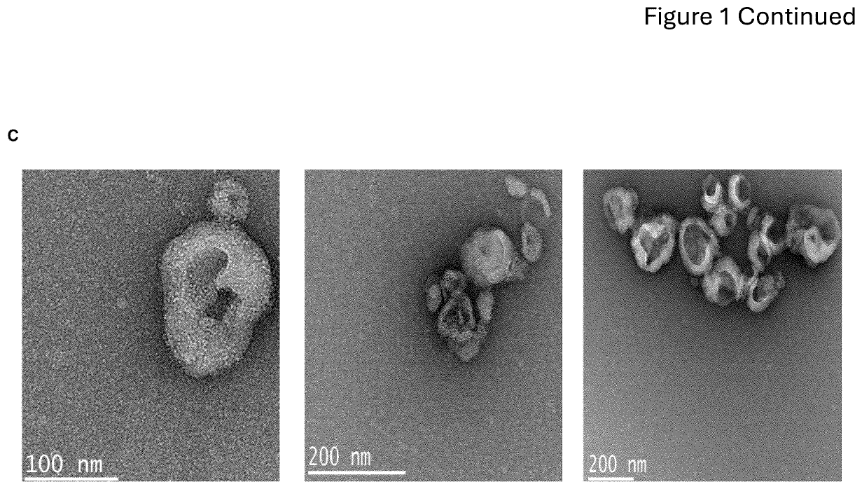

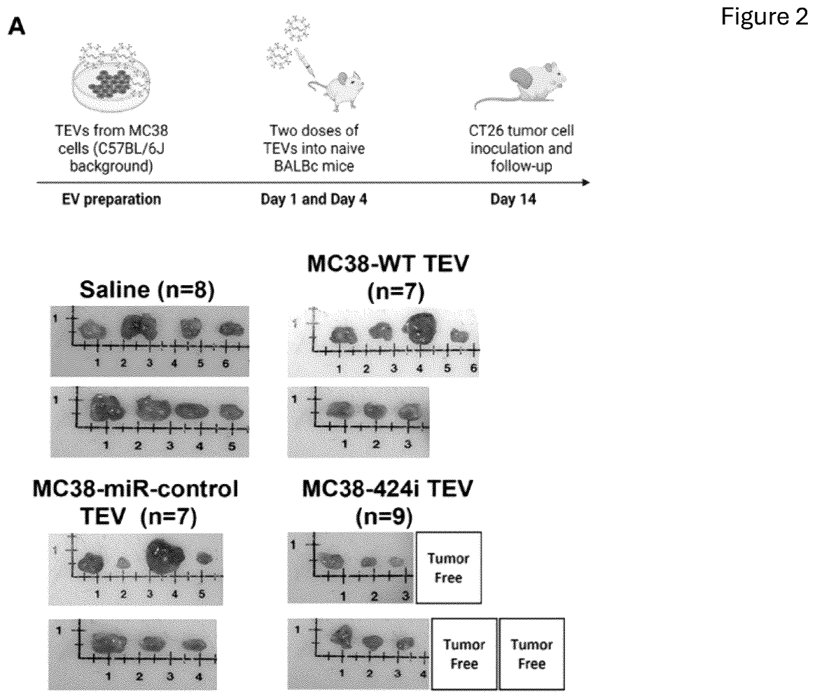

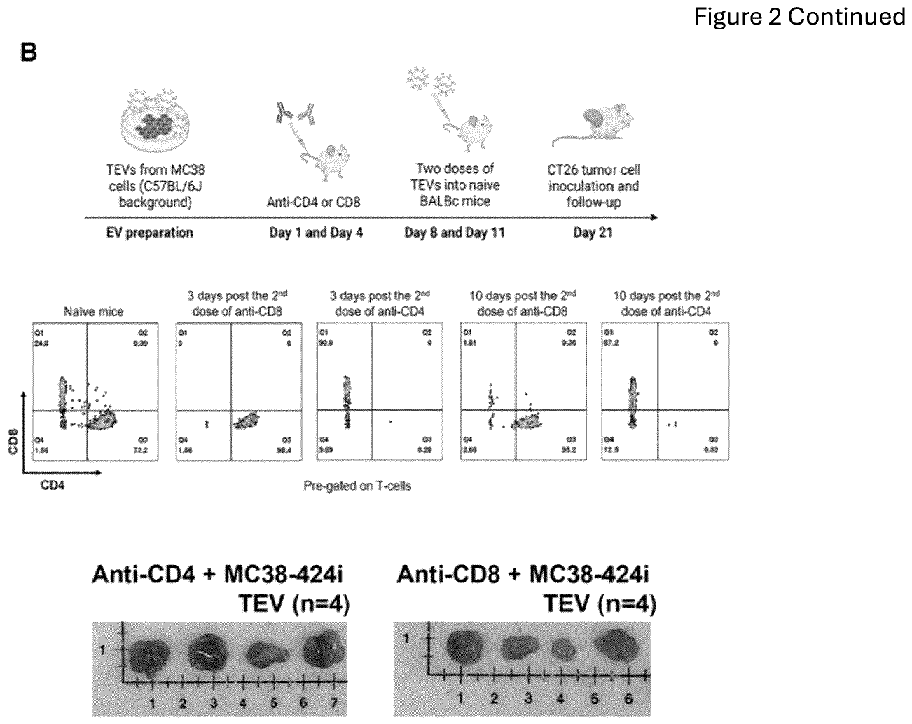

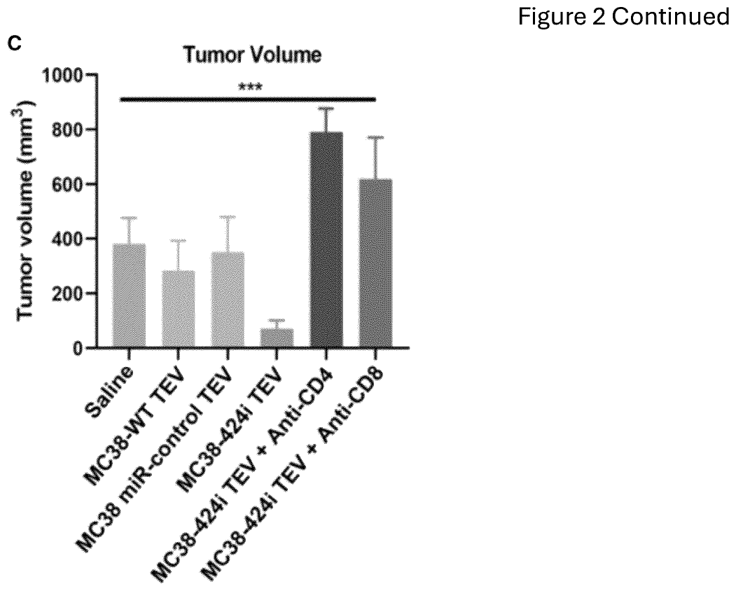

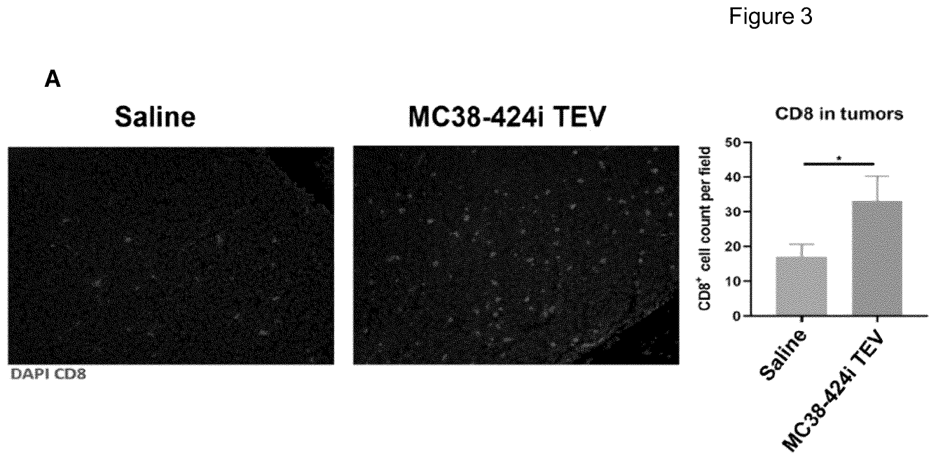

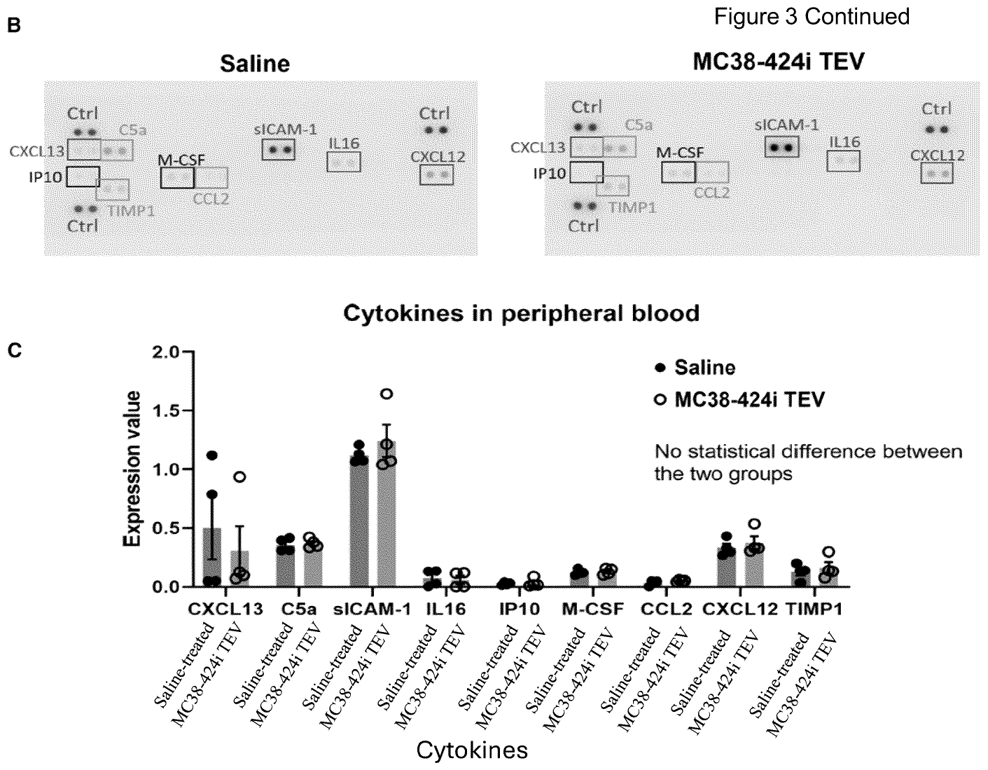

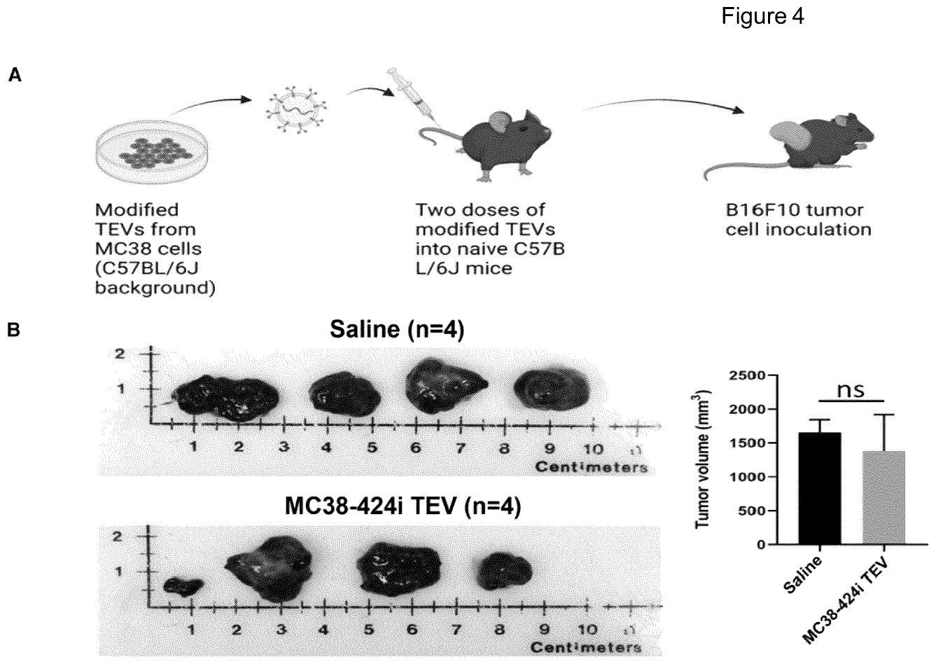

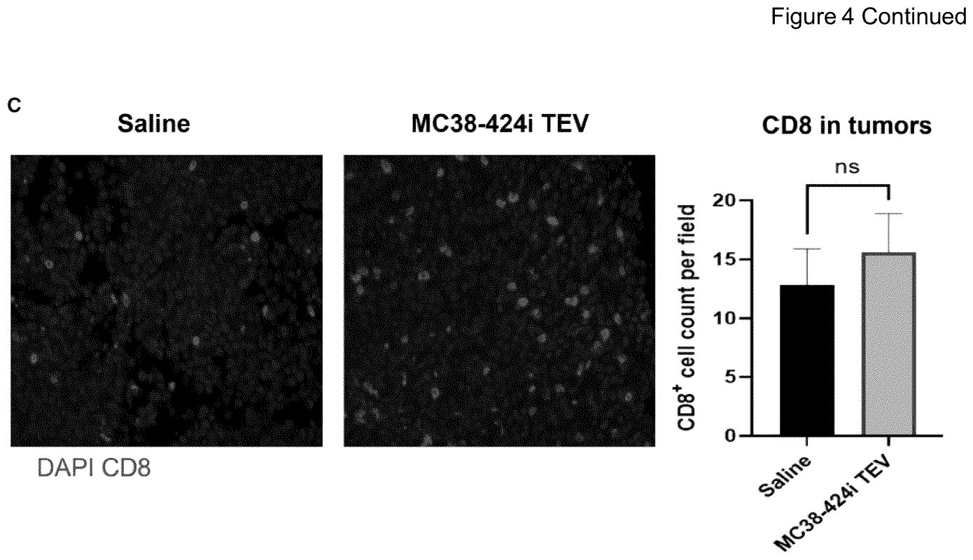

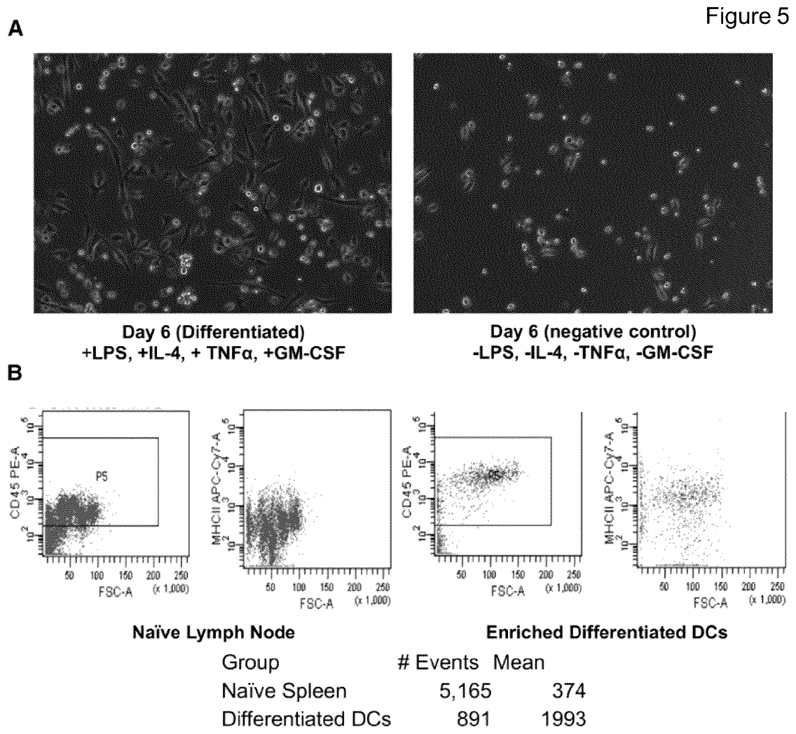

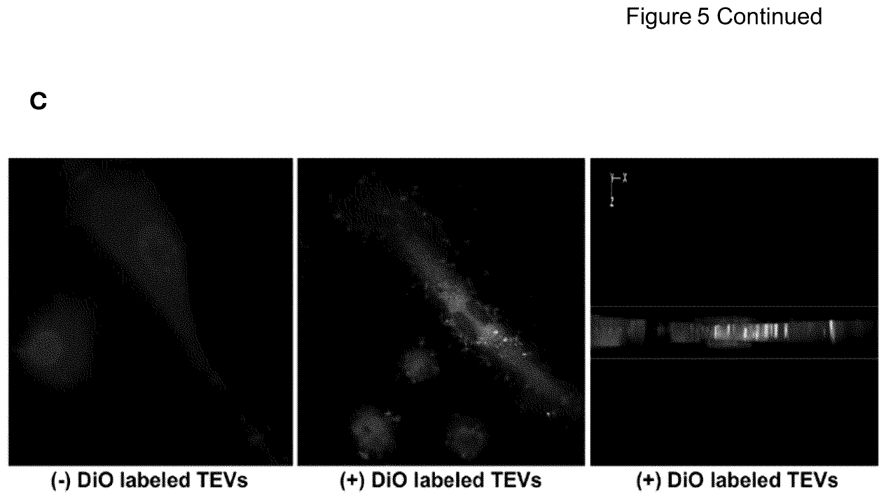

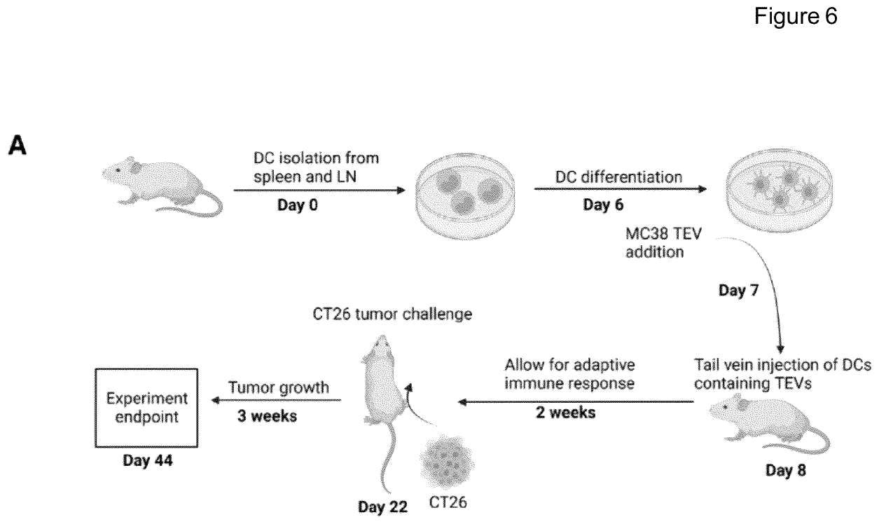

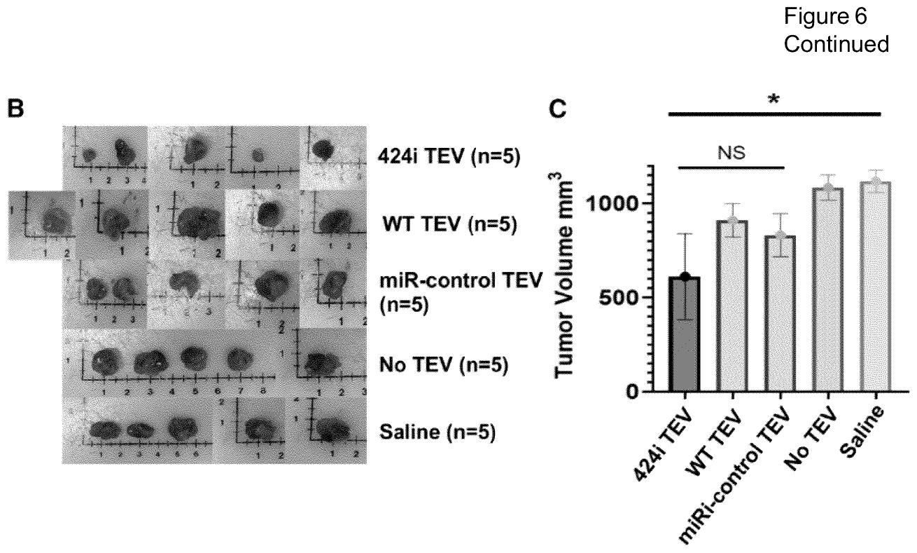

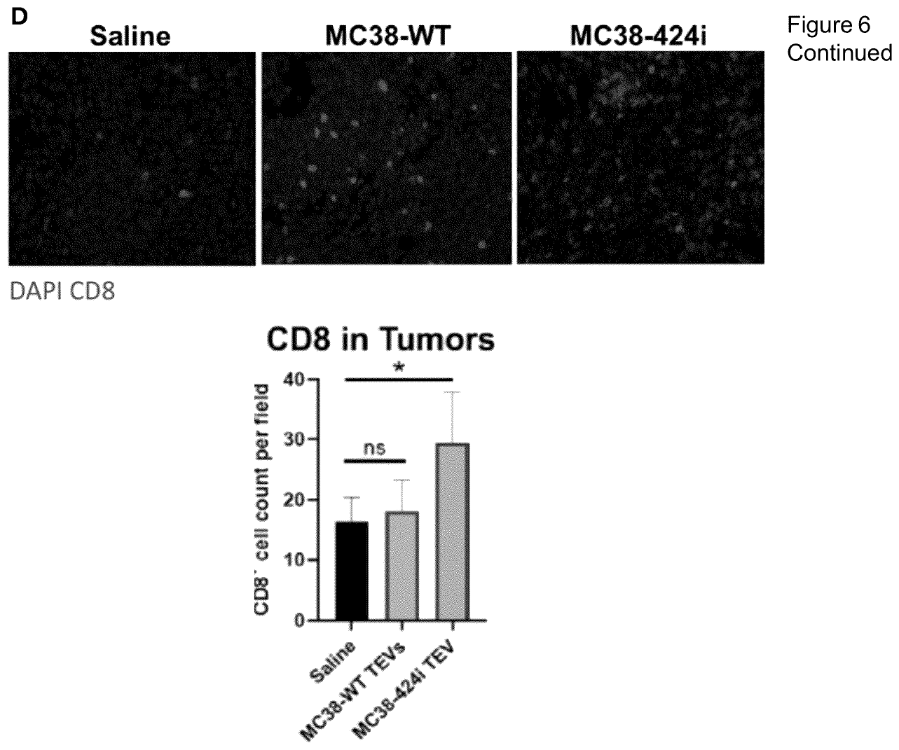

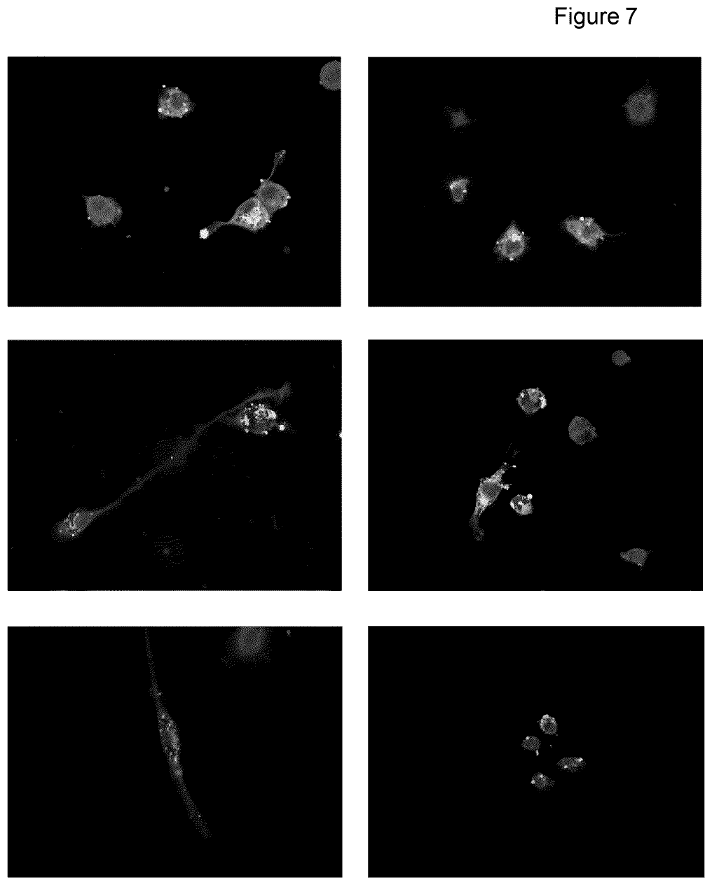

In addition, our investigation aimed to establish the capacity of DCs to capture TEVs and whether in-vitro-differentiated DCs could be pulsed with allogeneic MC38 TEVs, subsequently enabling their autologous transfer back into BALB/c mice to initiate an anti-tumor immune response in CT26 tumors. This paradigm enables the delivery of autologous tumor antigens without directly administering TEVs into the bloodstream. Convincing evidence emphasizes the role of antigen-presenting cells (APCs), such as DCs, as potent orchestrators of efficacious anti-tumor responses by presenting antigens to T cells.35 Previous studies have explored the exposure of tumor cells and antigens to DCs (exemplified by Sipuleucel-T) and revealed varied levels of efficacy in the context of prostate cancer.26,36,37 In this context, we postulated that the abundance of endogenous TEVs harboring miR-424, as demonstrated in our prior investigation, 19 might still impede the effective translation of DC vaccine strategies into clinical applications. Our data suggest that modified allogeneic TEVs can be successfully pulsed onto DCs in vitro (FIG. 5C). Additional images confirming TEV uptake can be referenced in FIG. 7.

Moreover, the autologous transfer of DCs to BALB/c mice demonstrated their potential in conferring protection against tumor growth, as evidenced by comparisons with the no TEVs and saline groups (FIGS. 6A-6C). While noticeable differences in tumor volumes within the allogeneic MC38 TEV groups were not observed, a striking contrast emerged regarding CD8+T cell infiltration. Specifically, DCs pulsed with allogeneic MC38-424i TEVs showed a significant distinction in CD8⁺ T cell infiltration compared with MC38-WT TEV. Our findings hold significant implications in the context of advanced tumors, which are often intrinsically immunosuppressive and unresponsive to ICIs due to complex mechanisms limiting functional CD8⁺ T cell infiltration.^{38,39} Our data signify the potential of DCs loaded with allogeneic MC38-424i TEVs in promoting CD8⁺ T cell infiltration into tumors (FIG. 6D).

An additional component we explored was the capacity of MC38-424i TEVs, when loaded onto DCs, to elicit anti-tumor immune responses, thereby circumventing the direct administration of TEVs into the bloodstream of BALB/c mice (FIG. 5C). Given the promising out-comes, it would be worthwhile for future investigations to incorporate the autologous transfer of DCs pulsed with MC38-424i TEVs. This endeavor would aim to ascertain whether ICI efficacy in CRC preclinical models could be influenced before the clinical translation phase.

Although our study reveals differences in tumor growth and immune response between CT26 and B16-F10 tumors following MC38-424i TEV administration, the underlying mechanisms responsible for these disparities are not fully elucidated. Further investigations addressing the potential molecular, cellular, and microenvironmental factors influencing these contrasting responses are essential to provide a comprehensive understanding.

Moreover, our study predominantly centers on CD8⁺ T cell infiltration and tumor growth, with limited exploration of other immune components that could contribute to the observed outcomes. A broader profiling of the immune landscape, encompassing various immune cell subsets, cytokine profiles, and immunosuppressive factors, would provide a more comprehensive understanding of the immune dynamics influenced by allogeneic TEVs.

In conclusion, this study underscores the protective potential of prophylactically administered allogeneic MC38 TEVs, lacking functional miR-424, against CT26 tumor growth facilitated by eliciting anti-tumor immune responses. Our investigations also reveal the tolerability of allogeneic MC38-424i TEVs, as they did not trigger significant changes in peripheral blood cytokine expression. Moreover, we established the feasibility of loading allogeneic MC38 TEVs onto DCs. Notably, the autologous transfer of modified TEV-pulsed DCs to mice demonstrated protective effects against CT26 tumor challenges, resulting in elevated CD8⁺ T cell infiltrates compared with MC38-WT TEVs and saline.

These findings warrant further investigation into the synergy between modified TEV-pulsed DCs and ICIs with orthotopic preclinical models of CRC. Experiments integrating a repertoire of immunotherapies designed to activate DCs in conjunction with ICIs are prerequisites before the translation and clinical implementation phases.

### Materials and Methods:

### Mice and Animal Husbandry

All animal studies were approved by the Institutional Animal Care and Use Committee (IACUC) of the University of Minnesota. All mice were housed in specific pathogen-free conditions with fully autoclaved cages to minimize non-tumor-specific immune activation. BALB/c and C57BL/6 mice were purchased from The Jackson Laboratory. Mice were bred in-house and were used for experiments between age 6 and 8 weeks.

### Cell Lines and Cell Culture

Mouse CRC cell line CT26 (ATCC CRL-2638) was purchased from ATCC. Dr. Nicholas Haining kindly provided mouse CRC cell line MC38. MC38 cells stably expressing miR322 inhibitor mouse homolog to miR-424 (MC38-424i) and MC38-miR-control cells were used in this study as described by Zhao et al.¹⁹ CT26 cells were cultured in complete Roswell Park Memorial Institute Medium (RPMI) 1640 (Gibco), supplemented with 10% heat-inactivated fetal bovine serum (FBS) (Thermo Fisher Scientific), 100 IU/mL penicillin, and 100 mg/mL streptomycin (Invitrogen Life Technologies). MC38-WT, MC38-424i, and MC38-miR-control cells were cultured in the complete Dulbecco's modified Eagle's medium (DMEM) (Gibco), supplemented with 10% heat-inactivated FBS, 100 IU/mL penicillin, and 100 mg/mL streptomycin. B16-F10 melanoma cells (ATCC CRL-6475) were purchased from ATCC and were cultured in the complete DMEM (Gibco), supplemented with 10% heat-inactivated FBS, 100 IU/mL penicillin, and 100 mg/mL streptomycin. Cell lines were authenticated and routinely tested for mycoplasma.

### Isolation of TEVs

Twenty-four hours before TEV isolation, cell medium was changed to DMEM supplemented with 10% exosome-depleted FBS, 100 IU/mL penicillin, and 100 mg/mL streptomycin. A standardized differential centrifugation protocol was used to purify TEVs from cell culture supernatants from MC38-WT, MC38-424i, and MC38-miR-control cells. Cell culture supernatants were centrifuged at 300×g for 10 min to remove cells. Supernatants were centrifuged for 3,000×g for 10 min to remove dead cells. Supernatants were centrifuged at 10,000×g for 30 min to remove cellular debris. Supernatants were centrifuged at 2,000×g for 30 min in Amicon Ultra-15 (Milli-pore) to concentrate supernatant. Supernatants were ultracentrifuged at 100,000×g for 70 min at 4 C with a SW40Ti rotor (Beckman Coulter). Pelleted TEVs were suspended and washed in PBS and underwent ultracentrifugation at 100,000×g for 70 min at 4 C. TEVs were collected in PBS for downstream analysis and experimentation.

### Characterization of Tumor Extracellular Vesicles

To characterize the purified TEVs, we first used electron microscopy. TEVs suspended in PBS were placed on Formvar carbon-coated nickel grids. TEVs on grids were stained with 2% uranyl acetate and allowed to air dry. TEVs were visualized using an FEI Tecnai G2 F30 Field Emission Gun Transmission Electron Microscope with a 4k x 4k ultrascan charge-coupled device camera. The size distributions and concentration of TEVs isolated from cell culture supernatants were determined using the NanoSight LM-10 microscope (Malvern Instruments) equipped with particle tracking software. Ten independent microscopic fields were captured and analyzed per cell line sample. Data were merged and presented as a single histogram plot. In addition, we tested TEV-related protein markers from isolated TEVs using western blotting. TEV protein concentration was estimated by using the Pierce Micro BCA Protein Assay Kit. Ten micrograms of TEV protein from MC38 and modified cell lines were loaded on SDS gels. Primary antibodies binding markers: CD81 (1:1,000, BioLegend, cat. no. 104902) and ALIX (1:2,000, BioLegend, cat. no. 634502) were used to validate TEV protein markers. b-Actin (1:1,500, Cell Signaling, cat. no. 8H10D10) and b-tubulin (1:2,000, Invitrogen, cat. no. MA5 16308) confirmed no cellular contaminates.

Prophylactic modified MC38 TEVs administration in CT26 and B16-F10 Subcutaneous Tumor Models

Ten micrograms of MC38-WT, MC38-424i, and MC38 miR-control TEVs suspended in sterile saline control were prophylactically injected twice into the tail vein of BALB/c or C57BL/6 mice, depending on the experimental setup. We allowed 2 weeks for an adaptive immune response before CT26 or B16-F10 tumor cell challenge. To establish subcutaneous tumors, we injected 2×10⁵ CT26 colon cancer cells suspended in 100 mL of 50:50 RPMI: Matrigel (Corning) into the right flank of BALB/c mice. Furthermore, 2 10⁵ B16-F10 melanoma cells were injected in the same preparation into the right flank of C57BL/6 mice. After tumor cell inoculation, tumors were measured 3 times per week using an electronic caliper. Tumor volumes were calculated using the formula (volume=[width² length]/2). Mice were sacrificed at 21 days following tumor inoculation. Tumor tissues were excised, imaged, and fixed in 10% neutral buffered formalin overnight. Fixed tissues were paraffin-embedded and sectioned in the University of Minnesota Clinical and Translational Sciences Institute. Mouse peripheral cytokines were measured using the Mouse Proteome Profiler Kit (R&D Systems) following the manufacturer's protocol on 100 mL of mouse serum from whole blood.

### Immunofluorescence of Tumor Tissues

Formalin-fixed paraffin-embedded tissues were deparaffinized with three xylene washes, rehydrated with gradient ethanol, and under-went antigen retrieval in antigen retrieval buffer (AR9, Perkin Elmer) in a 95 C water bath. Sections were blocked in 5% bovine serum albumin buffer for 30 min. Primary anti-mouse antibody CD8 (1:100, Abcam, cat. no. ab217344) was added and incubated overnight at 4 C. The tissue sections were washed twice with PBS and incubated with secondary antibodies (goat-anti-rat-A568, 1:250, Invitrogen, cat. no. A11077) and (goat-anti-rabbit-A568, 1:250, Invitrogen, cat. no. A11011) for 1 h. Tissues were washed twice with PBS, and slides were mounted with slide mounting medium with DAPI (Abcam, cat. no. ab104139). Slides were imaged on the BZX810 fluorescence microscope (Keyence). Quantitative image analysis was performed by counting positive signal percentage and fluorescence intensity signal in at least five randomly selected fields of each tumor tissue core.

CD4⁺ and CD8⁺ T cell Depletion

T cell subsets were depleted by intraperitoneally administering 400 mg of depleting antibody twice before prophylactic administration of MC38 TEVs and CT26 tumor challenge. CD4 T cells were depleted with anti-CD4 mAb (Clone GK1.5, Bio X Cell). CD8 T cells were depleted with anti-CD8a (Clone 2.43, Bio X Cell). CD4 and CD8 T cell depletion were confirmed using flow cytometry on the BD FACS CantoII (BD Biosciences) from the mouse spleen, lymph node, and peripheral blood. Antibodies for flow cytometry were CD3-APC (1:200, BioLegend, cat. no. 100236), CD8-APC-Cy7 (1:100, BioLegend, cat. no. 100714), CD4-BV510 (1:100, BioLegend, cat. no. 100449), CD11b-FITC

### Monocyte Isolation and Dendritic Cell Differentiation

According to the manufacturer's protocol, the monocytes were isolated using a negative selection from the mouse spleen, inguinal, axil-ary, and brachial lymph nodes with the Dynabeads Mouse DC Enrichment Kit (Invitrogen, cat. no. 11429D). Monocytes were plated at 10⁷ cells/well and differentiated using a Dendritic Cell Differentia-tion Kit (R&D Systems, cat. no. CDK004) according to the manufacturer's instructions. DCs were differentiated for 6 days in the kit's medium supplemented with 250 IU/mL IL-4 and 800 IU/mL granulocyte macrophage colony-stimulating factor for 2 days. Then the cells were centrifuged and cultured in fresh complete medium supplemented with 2,000 IU/mL IL-4 and 2,000 IU/mL granulocyte macrophage colony-stimulating factor. On day 6, cells were centrifuged and resus-pended in medium supplemented with 2,000 IU/mL IL-6, 400 IU/mL IL-1b, 2,000 IU/mL TNF-α, and 100 ng/ml lipopolysaccharide, and were cultured for a further 24 h. On day 7, we confirmed DC differentiation with flow cytometry to see the percentage of cells expressing a high level of MHC class II. Antibodies in flow cytometry were CD3-FITC (1:100, BioLegend, cat. no.100204), CD19-FITC (1:100, BioLegend, cat. no.115506), Nk1.1-FITC (1:100 BioLegend, cat. no. 108706), CD45-PE (1:200, BioLegend, cat. no. 103106), CD11b-APC (1:100, BioLegend, cat. no. 101212), IA/12E-APC-Cy7 (1:100, BioLegend, cat. no. 107628), and CD11c-BV510 (1:100, BioLegend, cat. no. 117338).

### Dendritic Cell TEV-Uptake Experiment

DCs and TEVs were isolated as previously described. DCs were plated on Labtek II chambers with chamber protectors coated with fibronectin (5 mg/mL) and differentiated in the same manner as previously described. TEVs from MC38 cell lines were stained with DiO lipophilic dye (Invitrogen) for 30 min and washed three times with PBS and ultracentrifugation at 100,000×g for 70 min at 4 C. TEV pellets labeled with DiO were suspended in DC medium, and 1 mg TEVs were added to DC cultures on day 6 with 100 ng/mL LPS. Following 24 h to allow for TEV uptake, DC culture on glass slides was stained with Cytopainter Red (Abcam, cat. no. ab219942) for 30 min according to the manufacturer's instructions and fixed with 4% formaldehyde for 30 min and washed 5 times with PBS. Chamber protectors were removed, slides were mounted with mounting medium with DAPI, and DCs exposed to MC38 TEVs were imaged on the BZX810 fluorescence microscope (Keyence). A series of photos were taken with a (4.4 mm) stepwise increase (0.4 mm) at each step on the z axis to validate the intracellular uptake of TEVs prior to the in vivo experiment.

### Prophylactic Autologous TEV-Dendritic Cell Animal Model

DCs were isolated and differentiated as previously described from BALB/c mice. TEVs from MC38-WT, MC38 322i, and MC38 miRi-control were isolated as described previously. On day 6, 10 mg of TEVs from the MC38 cell lines were administered to DCs in culture. On day 7, DCs were scraped from 10 cm³ dishes and counted and dosed at 1×10⁶ cells/injection in 100 mL of sterile PBS. Five groups (n=5/group) of animals were injected prophylactically with intravenous tail vein injection of autologous DC cell suspensions of (MC38-WT-DCs, MC38 322i-DCs, MC38 miRi-control-DCs, No TEV control-DCs, and saline. BALB/c mice underwent 2×10⁵ CT26 subcutaneous tumor challenge 14 days following the administration of DCs. Tumors were measured 3 times a week for 21 days using an electronic caliper. Mice were sacrificed at 21 days following tumor inoculation. Tumor tissues were excised, imaged, and fixed in 10% neutral buffered formalin. Fixed tissues were paraffin embedded and sectioned in the University of Minnesota Clinical and Translational Sciences Institute.

### Statistics

We used GraphPad Prism, versions 6.0 and 8.0, to perform statistical analyses and visualize data. We used the Student's t test to compare the treatment and control arms. One-way ANOVA was used when comparing more than two groups. All data are plotted as the mean+standard error of the mean (SEM). All statistics were evaluated at two-tailed a=0.05 unless otherwise corrected for multiple comparisons.

### REFERENCES

1. Siegel, R. L., Miller, K. D., Wagle, N. S., and Jemal, A. (2023). Cancer statistics, 2023. CA. Cancer J. Clin. 73, 17-48. doi.org/10.3322/caac.21763.
2. Larkin, J., Chiarion-Sileni, V., Gonzalez, R., Grob, J. J., Rutkowski, P., Lao, C. D., Cowey, C. L., Schadendorf, D., Wagstaff, J., Dummer, R., et al. (2019). Five-Year Survival with Combined Nivolumab and Ipilimumab in Advanced Melanoma. N. Engl. J. Med. 381, 1535-1546. doi.org/10.1056/NEJMoa1910836.
3. Motzer, R. J., Escudier, B., McDermott, D. F., George, S., Hammers, H. J., Srinivas, S., Tykodi, S. S., Sosman, J. A., Procopio, G., Plimack, E. R., et al. (2015). Nivolumab versus Everolimus in Advanced Renal-Cell Carcinoma. N. Engl. J. Med. 373, 1803-1813. doi.org/10.1056/NEJMoa1510665.
4. Hellmann, M. D., Paz-Ares, L., Bernabe Caro, R., Zurawski, B., Kim, S. W., Carcereny Costa, E., Park, K., Alexandru, A., Lupinacci, L., de la Mora Jimenez, E., et al. (2019). Nivolumab plus Ipilimumab in Advanced Non-Small-Cell Lung Cancer. N. Engl. J. Med. 381, 2020-2031. doi.org/10.1056/NEJMoa1910231.
5. Le, D. T., Uram, J. N., Wang, H., Bartlett, B. R., Kemberling, H., Eyring, A. D., Skora, A. D., Luber, B. S., Azad, N. S., Laheru, D., et al. (2015). PD-1 Blockade in Tumors with Mismatch-Repair Deficiency. N. Engl. J. Med. 372, 2509-2520. doi.org/10.1056/NEJMoa1500596.
6. Le, D. T., Durham, J. N., Smith, K. N., Wang, H., Bartlett, B. R., Aulakh, L. K., Lu, S., Kemberling, H., Wilt, C., Luber, B. S., et al. (2017). Mismatch repair deficiency pre-dicts response of solid tumors to PD-1 blockade. Science 357, 409-413. doi. org/10.1126/science.aan6733.
7. André, T., Shiu, K.-K., Kim, T. W., Jensen, B. V., Jensen, L. H., Punt, C., Smith, D., Garcia-Carbonero, R., Benavides, M., Gibbs, P., et al. (2020). Pembrolizumab in Microsatellite-Instability-High Advanced Colorectal Cancer. N. Engl. J. Med. 383, 2207-2218. doi.org/10.1056/NEJMoa2017699.
8. Boland, C. R., and Goel, A. (2010). Microsatellite instability in colorectal cancer. Gastroenterology 138, 2073-2087.e3. doi.org/10.1053/j.gastro.2009.12.064.
9. Mandal, R., Samstein, R. M., Lee, K. W., Havel, J. J., Wang, H., Krishna, C., Sabio, E. Y., Makarov, V., Kuo, F., Blecua, P., et al. (2019). Genetic diversity of tumors with mismatch repair deficiency influences anti-PD-1 immunotherapy response. Science 364, 485-491. doi.org/10.1126/science.aau0447.
10. Cercek, A., Lumish, M., Sinopoli, J., Weiss, J., Shia, J., Lamendola-Essel, M., El-Dika, I. H., Segal, N., Shcherba, M., Sugarman, R., et al. (2022). PD-1 Blockade in Mismatch Repair-Deficient, Locally Advanced Rectal Cancer. N. Engl. J. Med. 386, 2363-2376. doi.org/10.1056/NEJMoa2201445.
11. Pagès, F., Mlecnik, B., Marliot, F., Bindea, G., Ou, F. S., Bifulco, C., Lugli, A., Zlobec, I., Rau, T. T., Berger, M. D., et al. (2018). International validation of the consensus Immunoscore for the classification of colon cancer: a prognostic and accuracy study. The Lancet 391, 2128-2139. doi.org/10.1016/S0140-6736 (18) 30789-X.
12. Angell, H. K., Bruni, D., Barrett, J. C., Herbst, R., and Galon, J. (2020). The Immunoscore: Colon Cancer and Beyond. Clin. Cancer Res. 26, 332-339. doi.org/10.1158/1078-0432.Ccr-18-1851.
13. Sinicrope, F. A. (2022). Increasing Incidence of Early-Onset Colorectal Cancer. N. Engl. J. Med. 386, 1547-1558. doi.org/10.1056/NEJMra2200869.
14. Akimoto, N., Ugai, T., Zhong, R., Hamada, T., Fujiyoshi, K., Giannakis, M., Wu, K., Cao, Y., Ng, K., and Ogino, S. (2021). Rising incidence of early-onset colorectal cancer a call to action. Nat. Rev. Clin. Oncol. 18, 230-243. doi.org/10.1038/s41571-020-00445-1.
15. Kalluri, R., and LeBleu, V. S. (2020). The biology, function, and biomedical applications of exosomes. Science 367, eaau6977. doi.org/10.1126/science.aau6977.
16. Prakash, A., Gates, T., Zhao, X., Wangmo, D., and Subramanian, S. (2023). Tumor-derived extracellular vesicles in the colorectal cancer immune environment and immunotherapy. Pharmacol. Ther. 241, 108332. doi.org/10.1016/j.pharm-thera.2022.108332.
17. Chen, G., Huang, A. C., Zhang, W., Zhang, G., Wu, M., Xu, W., Yu, Z., Yang, J., Wang, B., Sun, H., et al. (2018). Exosomal PD-L1 contributes to immunosuppression and is associated with anti-PD-1 response. Nature 560, 382-386. doi.org/10.1038/s41586-018-0392-8.
18. Poggio, M., Hu, T., Pai, C. C., Chu, B., Belair, C. D., Chang, A., Montabana, E., Lang, U. E., Fu, Q., Fong, L., and Blelloch, R. (2019). Suppression of Exosomal PD-L1 Induces Systemic Anti-tumor Immunity and Memory. Cell 177, 414-427.e13. doi.org/10.1016/j.cell.2019.02.016.
19. Zhao, X., Yuan, C., Wangmo, D., and Subramanian, S. (2021). Tumor-Secreted Extracellular Vesicles Regulate T-Cell Costimulation and Can Be Manipulated To Induce Tumor-Specific T-Cell Responses. Gastroenterology 161, 560-574.e11. doi.org/10.1053/j.gastro.2021.04.036.
20. Horrevorts, S. K., Stolk, D. A., van de Ven, R., Hulst, M., van Het Hof, B., Duinkerken, S., Heineke, M. H., Ma, W., Dusoswa, S. A., Nieuwland, R., et al. (2019). Glycan-Modified Apoptotic Melanoma-Derived Extracellular Vesicles as Antigen Source for Anti-Tumor Vaccination. Cancers 11, 1266.
21. Wolfers, J., Lozier, A., Raposo, G., Regnault, A., Théry, C., Masurier, C., Flament, C., Pouzieux, S., Faure, F., Tursz, T., et al. (2001). Tumor-derived exosomes are a source of shared tumor rejection antigens for CTL cross-priming. Nat. Med. 7, 297-303. doi.org/10.1038/85438.
22. Diamond, J. M., Vanpouille-Box, C., Spada, S., Rudqvist, N. P., Chapman, J. R., Ueberheide, B. M., Pilones, K. A., Sarfraz, Y., Formenti, S. C., and Demaria, S. (2018). Exosomes Shuttle TREX1-Sensitive IFN-Stimulatory dsDNA from Irradiated Cancer Cells to DCs. Cancer Immunol. Res. 6, 910-920. doi.org/10.1158/2326-6066.Cir-17-0581.
23. Samuel, M., and Gabrielsson, S. (2021). Personalized medicine and back-allogeneic exosomes for cancer immunotherapy. J. Intern. Med. 289, 138-146. doi.org/10.1111/joim.12963.
24 Larssen, P., Veerman, R. E., Akpinar, G. G., Hiltbrunner, S., Karlsson, M. C. I., and Gabrielsson, S. (2019). Allogenicity Boosts Extracellular Vesicle-Induced Antigen-Specific Immunity and Mediates Tumor Protection and Long-Term Memory In Vivo. J. Immunol. 203, 825-834. doi.org/10.4049/jimmunol.1801628.
25. Goldszmid, R. S., Idoyaga, J., Bravo, A. I., Steinman, R., Mordoh, J., and Wainstok, R. (2003). Dendritic cells charged with apoptotic tumor cells induce long-lived protective CD4⁺ and CD8⁺ T cell immunity against B16 melanoma. J. Immunol. 171, 5940-5947. doi.org/10.4049/jimmunol.171.11.5940.
26. Kantoff, P. W., Higano, C. S., Shore, N. D., Berger, E. R., Small, E. J., Penson, D. F., Redfern, C. H., Ferrari, A. C., Dreicer, R., Sims, R. B., et al. (2010). Sipuleucel-T Immunotherapy for Castration-Resistant Prostate Cancer. N. Engl. J. Med. 363, 411-422. doi.org/10.1056/NEJMoa1001294.
27. Wooster, A. L., Girgis, L. H., Brazeale, H., Anderson, T. S., Wood, L. M., and Lowe, D. B. (2021). Dendritic cell vaccine therapy for colorectal cancer. Pharmacol. Res. 164, 105374. doi.org/10.1016/j.phrs.2020.105374.
28. Burbage, M., Rocañín-Arjó, A., Baudon, B., Arribas, Y. A., Merlotti, A., Rookhuizen, D. C., Heurtebise-Chrétien, S., Ye, M., Houy, A., Burgdorf, N., et al. (2023). Epigenetically controlled tumor antigens derived from splice junctions between exons and transposable elements. Sci. Immunol. 8, eabm6360. doi.org/10.1126/sciimmunol.abm6360.
29. Dunn, G. P., Bruce, A. T., Ikeda, H., Old, L. J., and Schreiber, R. D. (2002). Cancer immunoediting: from immunosurveillance to tumor escape. Nat. Immunol. 3, 991-998. https://doi.org/10.1038/ni1102-991.
30. Dunn, G. P., Old, L. J., and Schreiber, R. D. (2004). The three Es of cancer immunoediting. Annu. Rev. Immunol. 22, 329-360. doi.org/10.1146/annurev.immunol. 22.012703.104803.
31. Spranger, S., Luke, J. J., Bao, R., Zha, Y., Hernandez, K. M., Li, Y., Gajewski, A. P., Andrade, J., and Gajewski, T. F. (2016). Density of immunogenic antigens does not explain the presence or absence of the T-cell-inflamed tumor microenvironment in melanoma. Proc. Natl. Acad. Sci. USA 113, E7759-E7768.doi.org/10. 1073/pnas.1609376113.
32. Ho, W. W., Gomes-Santos, I. L., Aoki, S., Datta, M., Kawaguchi, K., Talele, N. P., Roberge, S., Ren, J., Liu, H., Chen, I. X., et al. (2021). Dendritic cell paucity in mismatch repair-proficient colorectal cancer liver metastases limits immune check-point blockade efficacy. Proc. Natl. Acad. Sci. USA 118, e2105323118. doi.org/10.1073/pnas.2105323118.
33. Tvedt, T. H. A., Vo, A. K., Ø, B., and Reikvam, H. (2021). Cytokine Release Syndrome in the Immunotherapy of Hematological Malignancies: The Biology behind and Possible Clinical Consequences. J. Clin. Med. November 6, 10.doi.org/10.3390/jcm10215190.
34. Abboud, R., Wan, F., Mariotti, J., Arango, M., Castagna, L., Romee, R., Hamadani, M., and Chhabra, S. (2021). Cytokine release syndrome after haploidentical hematopoi-etic cell transplantation: an international multicenter analysis. Bone Marrow Transpl. 56, 2763-2770. doi.org/10.1038/s41409-021-01403-w.
35. Marciscano, A. E., and Anandasabapathy, N. (2021/02/01/2021). The role of dendritic cells in cancer and anti-tumor immunity. Semin. Immunol. 52, 101481. oi. org/10.1016/j.smim.2021.101481.
36. Buonaguro, L., Petrizzo, A., Tornesello, M. L., and Buonaguro, F. M. (2011). Translating Tumor Antigens into Cancer Vaccines. Clin. Vaccin. Immunol. 18, 23-34. doi.org/10.1128/CVI.00286-10.
37. Small, E. J., Schellhammer, P. F., Higano, C. S., Redfern, C. H., Nemunaitis, J. J., Valone, F. H., Verjee, S. S., Jones, L. A., and Hershberg, R. M. (Jul. 1 2006). Placebo-controlled phase III trial of immunologic therapy with sipuleucel-T (APC8015) in patients with metastatic, asymptomatic hormone refractory prostate cancer. J. Clin. Oncol. 24, 3089-3094. doi.org/10.1200/jco.2005.04.5252.
38. Zhao, X., and Subramanian, S. (2017). Intrinsic Resistance of Solid Tumors to Immune Checkpoint Blockade Therapy. Cancer Res. 77, 817-822. doi.org/10.1158/0008-5472.Can-16-2379.
39. Morad, G., Helmink, B. A., Sharma, P., and Wargo, J. A. (Oct. 14 2021). Hallmarks of response, resistance, and toxicity to immune checkpoint blockade. Cell 184, 5309-5337. doi.org/10.1016/j.cell.2021.09.020.

What is claimed:

1. A composition comprising a dendritic cell (DC) contacted ex-vivo with an allogeneic extracellular vesicle (EV) to generate an EV loaded DC, wherein the EV has reduced or lacking expression of microRNA (miRNA)-424.

2. The composition of claim 1, wherein the EV are exosomes or microvesicles and are isolated from a tumor cell that has been modified to inhibit or reduce the expression of miRNA-424.

3. The composition of claim 2, wherein the tumor cell is a cultured tumor cell or tumor organoid.

4. The composition of claim 3, wherein the tumor cell is selected from the group consisting of colorectal cancer cell, breast cancer cell, endometrial cancer cell, prostate cancer cell, lung cancer cell, melanoma cell and pancreatic cancer cell.

5. The composition of claim 1, wherein the DC is isolated or derived from a subject diagnosed with cancer.

6. The composition of claim 5, wherein the DC are contacted with allogeneic EV isolated from a tumor cell of the same cancer type as the subject with cancer.

7. The composition of claim 6, wherein the subject is diagnosed with a cancer comprising at least one of colorectal cancer, breast cancer, endometrial cancer, prostate cancer, lung cancer, melanoma or pancreatic cancer and the EV is isolated from an allogeneic tumor cell of the same cancer type.

8. The composition of claim 1, wherein the EV comprise additional cargo.

9. A method of making an EV loaded DC, the method comprising contacting a DC ex-vivo with allogeneic EV modified to reduce or lack expression of miRNA-424.

10. The method of claim 9, wherein the EV is isolated from a tumor cell that has been modified to inhibit or reduce the expression of miRNA-424, wherein the tumor cell is a cultured tumor cell or tumor organoid, and wherein the tumor cell is selected from the group comprising a colorectal cancer cell, breast cancer cell, endometrial cancer cell, prostate cancer cell, lung cancer cell, melanoma cell and pancreatic cancer cell.

11. The method of claim 9, wherein the DC is isolated or derived from a subject diagnosed with cancer, wherein the DC is contacted with an EV isolated from an allogeneic tumor cell of the same cancer type as the subject with cancer, and wherein the cancer in the subject comprises at least one of colorectal cancer, breast cancer, endometrial cancer, prostate cancer, lung cancer, melanoma or pancreatic cancer and the EV are isolated from a tumor cell of the same cancer type.

12. The method of claim 9, wherein the DC is contacted ex-vivo with allogeneic EV by a method selected from at least one of co-incubation, electroporation, sonication, freeze-thaw, and transfection.

13. The method of claim 9, wherein the exosomes comprise additional cargo.

14. A method of treating cancer, the method comprising:
a) isolating a DC from a subject diagnosed with cancer, or deriving a DC from a stem or progenitor cell isolated from a subject diagnosed with cancer;
b) isolating an EV from a tumor cell, wherein the tumor cell is allogeneic to the DC and of the same cancer type as the cancer diagnosed in the subject; wherein the EV has been modified to reduce or lack the expression of miR-424;
c) contacting the DC of step (a) with the isolated EV of step (b) ex-vivo to prepare EV loaded DCs; and
d) administering the EV loaded DC of step (c) to the subject diagnosed with cancer.

15. The method of claim 14, wherein the EV has been modified to comprise additional cargo, wherein the additional cargo is selected from the group consisting of proteins, anticancer drugs, small molecules and nucleic acids.

16. The method of claim 14, wherein the cancer comprises at least one of colorectal cancer, breast cancer, endometrial cancer, prostate cancer, lung cancer, melanoma or pancreatic cancer.

17. The method of claim 14, wherein at least 10⁶ loaded DC are administered to the subject, and wherein the loaded DC are administered intravenously, intratumorally, subcutaneously, or intraperitoneally.

18. A method of treating cancer or stimulating an anti-tumor response in a subject having cancer, the method comprising administering the composition of claim 1 to the subject.

19. The method of claim 18, further comprising administering an immune checkpoint inhibitor to the subject.

20. The method of claim 19, wherein the cancer is selected from the group consisting of colorectal cancer, breast cancer, endometrial cancer, prostate cancer, lung cancer, melanoma and pancreatic cancer and wherein the composition is administered intravenously, intratumorally, subcutaneously, or intraperitoneally.
